SEQUENCE LISTING (2 sequences)

SEQ ID NO: 1 - protein, 40 aa, Homo sapiens
DAEFRHDSGYEVHHQKLVFFAEDVGSNKGAIIGLMVGGVV

SEQ ID NO: 2 - protein, 42 aa, Homo sapiens [sequence omitted]

(19) United States
(12) Patent Application Publication
(10) Pub. No.: US 2009/0087916 A1
(43) Pub. Date: Apr. 2, 2009

(54) Assay method for identifying drug candidate

(75) Inventors: Naoyuki Sato, Hyogo (JP); Masayasu Okochi, Kyoto (JP); Yoshiaki Taniyama, Osaka (JP); Toshio Ogihara, Osaka (JP); Ryuichi Morishita, Osaka (JP)

Correspondence Address: BANNER & WITCOFF, LTD., 1100 13th STREET, N.W., SUITE 1200, WASHINGTON, DC, 20005-4051, US

(73) Assignee: AnGesMG, Inc., Osaka (JP)

(21) Appl. No.: 10/594,927

(22) PCT Filed: Mar. 30, 2005
(86) PCT No.: PCT/JP2005/006728

§ 371 Date: Dec. 21, 2006

(30) Foreign Application Priority Data
Mar. 31, 2004 (JP) 2004-107746

(51) Int. Cl. G01N 33/68; A61N 7/00; C07H 19/24; C07D 311/30; C07H 15/20; C07C 49/248; C07D 279/20; C07K 1/107

(52) U.S. Cl. 436/86; 601/2; 536/6.5; 549/400; 536/18.2; 568/329; 544/37; 530/345

(57) ABSTRACT

The present invention provides a method of identifying a drug candidate capable of removing peptide, oligopeptide, polypeptide or protein from fibril or aggregate, which includes measuring, in the presence of a test compound, the concentration of a soluble peptide, a soluble oligopeptide, a soluble polypeptide or a soluble protein in an equilibrium state in a solvent. Moreover, the present invention provides a dissolution promoter to remove peptide, oligopeptide, polypeptide or protein from fibril or aggregate, which contains the compound obtained by the identification method as an active ingredient.

### TECHNICAL FIELD

The present invention relates to a method of identifying a drug candidate capable of removing peptide, oligopeptide, polypeptide or protein from fibril or aggregate.

### BACKGROUND ART

Senile plaque containing amyloid β (hereinafter to be also referred to as Aβ) is one of the pathological features of Alzheimer's disease.

Reduction of Aβ is considered to be a primary therapeutic target.

There are reports on amyloid clearance after immunization with anti-Aβ antibodies.

Recent data reveal that the antibodies may act as peripheral sink for Aβ to alter the periphery/brain dynamics.

The factual disappearance of senile plaque indicates that the Aβ aggregation process is reversible.

However, there are no evidence of dissolution of soluble Aβ from aggregated Aβ.

Here, the present inventors have developed a sink-like Aβ assay for the analysis of in vitro transfer (replacement) of fibril to the supernatant following centrifugation, by which not only dissolution of soluble Aβ from aggregated Aβ but also promotion of the dissolution of Aβ by ultrasonication are indicated.

A compound or ultrasonication that promotes dissolution of Aβ in the in vitro sink-like Aβ assay may become a potential alternative to the treatment of Alzheimer's disease.

Alzheimer's disease (AD) is a neurodegenerative disease characterized by the presence of senile plaques in brain.

Aβ peptide consisting of 40-42 amino acids is the main component of senile plaque.

The inheritable character of this disease is related to the mutation of amyloid precursor protein (APP) and presenilin gene.

The mutation in these genes relating to the disease increases production of Aβ (1-42) predominantly present in the senile plaques.

Recently, immunization of mutant APP transgenic mouse with Aβ has been shown to be effective for suppressing plaque deposition (Schenk, Barbour et al. 1999).

Both plaques and neuritic lesions were reversible after administration of a single dose of Aβ antibody (Lombardo, Stern et al. 2003).

In addition to the Aβ antibody, a peripheral treatment with a pharmaceutical agent having high affinity for Aβ (gelsolin or GM1) reduced the level of Aβ in the brain (Matsuoka, Saito et al. 2003).

It has been proposed that generation of a peripheral sink mechanism via administration of antibodies is useful for treating a number of disorders characterized by the abnormal protein accumulation in an extracellular space (DeMattos, Bales et al. 2001).

The Aβ aggregation process has been sufficiently studied in vitro.

However, the reverse phenomenon wherein Aβ is dissolved from aggregated Aβ has not been studied yet.

In this study, we developed a novel in vitro sink-like Aβ assay to study dissolution of Aβ from aggregated Aβ.

We found that soluble Aβ dissolved from aggregated Aβ.

In addition, ultrasonication promoted dissolution of Aβ.

The compound and/or ultrasonication that promoted dissolution of Aβ in the in vitro sink-like Aβ assay may become an alternative for the treatment of Alzheimer's disease.

WO94/10569 (JP-A-8-502587) discloses a method for diagnosing or monitoring a β-amyloid peptide (hereinafter to be also referred to as βAP)-related condition in a patient, said method comprising: measuring the amount of soluble βAP or βAP fragments in a patient sample; comparing the measured amount with a predetermined amount of βAP or βAP fragments; and assessing patient status based on the difference between the measured and predetermined βAP or βAP fragment amounts (claim 23).

WO00/26238 (JP-A-2002-531065) discloses a method of identifying an agent which is capable of preventing, reducing and/or reversing the conversion of prion protein to a β-form, the method comprising: providing a sample of a prion protein and comparing the amount of the β-form qualitatively or quantitatively in the presence and absence of a test agent (claim 50).

WO00/43791 (JP-A-2002-540383) discloses a method comprising: forming a solution containing a species capable of binding neurodegenerative disease aggregate-forming or fibril-forming species and one of a sample suspected of containing neurodegenerative disease aggregate-forming or fibril-forming species for a drug candidate for inhibition of neurodegenerative disease aggregate or fibril-formation; and, without transferring any components into the solution or removing the solution from its container, detecting aggregation in the solution characteristic of neurodegenerative diseases (claim 172).

For evaluation of a number of test compounds, however, these prior arts are not easy, not quick, not highly reliable or not economical methods.

### DISCLOSURE OF THE INVENTION

The present inventors have conducted intensive studies in an attempt to establish a method of identifying a drug candidate usable for the treatment of a disease caused by aggregation of peptide, oligopeptide, polypeptide or protein. They have found that the Aβ aggregation process is reversible, and established a method capable of identifying a drug candidate capable of removing peptide, oligopeptide, polypeptide or protein from fibril or aggregate, by measuring, in the presence of a test compound, the concentration of a soluble peptide, a soluble oligopeptide, a soluble polypeptide or a soluble protein in an equilibrium state in a solvent.

Accordingly, the present invention provides the following.

(1) A method of identifying a drug candidate capable of removing peptide, oligopeptide, polypeptide or protein from fibril or aggregate, which comprises measuring, in the presence of a test compound, the concentration of a soluble peptide, a soluble oligopeptide, a soluble polypeptide or a soluble protein in an equilibrium state in a solvent.
(2) The method of the above-mentioned (1), wherein the drug candidate is used for the treatment of a disease caused by the aggregation of peptide, oligopeptide, polypeptide or protein.
(3) The method of the above-mentioned (1) or (2), wherein the disease is selected from the group consisting of Alzheimer's disease (AD), Parkinson's syndrome (PD), Huntington chorea, prion disease, Down's syndrome, Lewy body dementia, multiple system atrophy, Creutzfeldt-Jakob disease, Gerstmann-Sträussler syndrome, mad cow disease, spinobulbar muscular atrophy, spinocerebellar ataxia (SCA), dentatorubral-pallidoluysian atrophy (DRPLA), familial amyotropic lateral sclerosis retinitis, FTDP-17 (frontotemporal dementia and parkinsonisms linked to chromosome 17), progressive supranuclear palsy, corticobasal degeneration, Pick disease, familial British dementia and familial dementia accompanying neuroserpin inclusion bodies.
(4) The method of any of the above-mentioned (1) to (3), wherein the fibril or aggregate was formed in vitro.
(5) The method of any of the above-mentioned (1) to (4), wherein the equilibrium state is achieved under ultrasonication.
(6) The method of any of the above-mentioned (1) to (5), wherein the ultrasonication is substantially unaccompanied by heat generation.
(7) The method of any of the above-mentioned (1) to (6), wherein the ultrasonication conditions include 5 repeats of a 30 second ultrasonication at 1 MHz, 2 W/cm², duty ratio 20% with a 10 second pause.
(8) A method of identifying a drug candidate capable of removing p-amyloid (Aβ) from fibril or aggregate formed in vitro, which comprises measuring, in the presence of a test compound, the concentration of soluble β-amyloid (Aβ) in an equilibrium state in a solvent.
(9) The method of the above-mentioned (8), wherein the fibril or aggregate consists of Aβ (1-40).
(10) The method of the above-mentioned (8), wherein the fibril or aggregate consists of Aβ (1-42).
(11) The method of any of the above-mentioned (8) to (10), wherein the equilibrium state is achieved under ultrasonication.
(12) The method of any of the above-mentioned (8) to (11), wherein the ultrasonication is substantially unaccompanied by heat generation.
(13) The method of any of the above-mentioned (8) to (12), wherein the ultrasonication conditions include 5 repeats of a 30 second ultrasonication at 1 MHz, 2 W/cm², duty ratio 20% with a 10 second pause.
(14) A treatment method of a disease caused by aggregation of peptide, oligopeptide, polypeptide or protein, which comprises application of ultrasonication to a patient.
(15) The method of the above-mentioned (14), wherein the disease is selected from the group consisting of Alzheimer's disease, Parkinson's syndrome, Huntington chorea, prion disease, Down's syndrome, Lewy body dementia, multiple system atrophy, Creutzfeldt-Jakob disease, Gerstmann-Sträussler syndrome, mad cow disease, spinobulbar muscular atrophy, spinocerebellar ataxia (SCA), dentatorubral-pallidoluysian atrophy (DRPLA), familial amyotropic lateral sclerosis retinitis, FTDP-17, progressive supranuclear palsy, corticobasal degeneration, Pick disease, familial British dementia and familial dementia accompanying neuroserpin inclusion bodies.
(16) A dissolution promoter for removing peptide, oligopeptide, polypeptide or protein from fibril or aggregate, which comprises, as an active ingredient, the compound obtained by the method described in any of the above-mentioned (1) to (13).
(17) A dissolution promoter for removing peptide, oligopeptide, polypeptide or protein from fibril or aggregate, which comprises, as an active ingredient, at least one selected from the group consisting of ferric dehydroporphyrin IX, amphotericin B, myricetin, tannic acid, curcumin, azure B and basic blue 41.
(18) A dissolution method comprising dissolving peptide, oligopeptide, polypeptide or protein from fibril or aggregate, by the use of the compound obtained by the method described in any of the above-mentioned (1) to (13).
(19) Use of the compound obtained by the method described in any of the above-mentioned (1) to (13) for removing peptide, oligopeptide, polypeptide or protein from fibril or aggregate.
(20) An apparatus for treating a disease caused by the aggregation of peptide, oligopeptide, polypeptide or protein, which comprises a means for ultrasonicating an affected part to remove peptide, oligopeptide, polypeptide or protein from fibril or aggregate.
(21) A method of identifying a drug candidate capable of removing peptide, oligopeptide, polypeptide or protein from fibril or aggregate, which comprises measuring, in the presence of a test compound, the concentration of the soluble peptide, soluble oligopeptide, soluble polypeptide or soluble protein dissolved from fibril or aggregate in a solvent.

### BRIEF DESCRIPTION OF THE DRAWINGS

FIG. 1 shows experimental procedures of in vitro sink-like Aβ assay.

FIG. 2 shows dissolution of soluble Aβ from aggregated Aβ, as a result of sandwich ELISA for soluble Aβ (1-40) and Aβ (1-42).

FIG. 3 shows the results of mass analysis of dissolved Aβ (1-40) and Aβ (1-42).

FIG. 4 shows the results of Western blot analysis of dissolved Aβ (1-40).

FIG. 5 shows SEC analysis charts of dissolved Aβ (1-40), which show promoted dissolution of Aβ by ultrasonication.

FIG. 6 shows experimental procedures for ultrasonication.

FIG. 7 shows the results of sandwich ELISA of dissolved Aβ (1-40).

FIG. 8 shows SEC analysis charts of fibrillization and oligomerization of Aβ (1-42).

FIG. 9 is a schematic showing of the in vitro sink-like Aβ assay.

FIG. 10 shows the results of Example 2.

### DETAILED DESCRIPTION OF THE INVENTION

The present invention is explained in detail in the following.

### <Identification Method of Drug Candidates>

The present invention provides a method of identifying a drug candidate capable of removing peptide, oligopeptide, polypeptide or protein from fibril or aggregate, which comprises measuring, in the presence of a test compound, the concentration of a soluble peptide, a soluble oligopeptide, a soluble polypeptide or a soluble protein in an equilibrium state in a solvent.

In the step of the identification method of the present invention, the concentration of a soluble peptide, a soluble oligopeptide, a soluble polypeptide or a soluble protein in an equilibrium state in a solvent is measured in the presence of a test compound.

The test compound may be any known compound or novel compound and, for example, nucleic acid, carbohydrates, lipid, protein, peptide, organic low-molecular-weight compound, compound library prepared using combinatorial chemistry techniques, random peptide library prepared using solid phase synthesis or phage display method, natural components derived from microorganism, plants, animals, marine animals etc., and the like can be mentioned. Alternatively, the test compound may be one actually used for the treatment of a disease caused by aggregation of peptide, oligopeptide, polypeptide or protein.

The above-mentioned soluble peptide, oligopeptide, polypeptide and protein are not particularly limited as long as their aggregation can cause an adverse effect, and eventually a disease and, for example, Aβ, prion protein, polyglutamine, α synuclein, tau, superoxide dismutase-1 (SOD1), neuroserpin, amyloid precursor protein (APP) and the like can be mentioned.

Aβ is a main component constituting senile plaque, which is one of the major neuropathological changes in Alzheimer's disease, and produced by cleavage of amyloid precursor protein (APP) with two kinds of (β- and γ-) secretases. The major molecule species includes Aβ (1-40) (SEQ ID NO: 1) consisting of 40 amino acid residues and Aβ (1-42) (SEQ ID NO: 2) longer by 2 residues in the C-terminal side.

The aggregation or accumulation of Aβ is considered to cause diseases such as Alzheimer's disease, Down's syndrome and the like, and the accumulation sites thereof are senile plaque and cerebrovascular amyloid. Aggregation or accumulation of prion protein is considered to cause diseases such as Creutzfeldt-Jakob disease, Gerstmann-Sträussler syndrome, mad cow disease and the like, and the accumulation sites thereof are synapse, nerve cell and Kuru plaque amyloid. The aggregation or accumulation of polyglutamine is considered to cause diseases such as spinobulbar muscular atrophy, spinocerebellar ataxia (SCA), dentatorubral-pallidoluysian atrophy (DRPLA), Huntington chorea and the like, and the accumulation sites thereof are neuronal intranuclear inclusion bodies. The aggregation or accumulation of α-synuclein is considered to cause diseases such as Parkinson's syndrome, Lewy body dementia, multiple system atrophy and the like, and the accumulation sites thereof are Lewy body (intraneuronal) and oligodendrocyte (GCI). The aggregation or accumulation of tau is considered to cause diseases such as Alzheimer's disease, FTDP-17, progressive supranuclear palsy, corticobasal degeneration, Pick disease and the like, and the accumulation sites thereof are neurofibrillary tangle, glial inclusion bodies and Pick body. The aggregation or accumulation of superoxide dismutase-1 is considered to cause diseases such as familial amyotropic lateral sclerosis retinitis and the like, and the accumulation sites thereof are Lewy body-like inclusion bodies. The aggregation or accumulation of neuroserpin is considered to cause diseases such as familial dementia accompanying neuroserpin inclusion bodies and the like, and the accumulation sites thereof are Collins body (intraneronal). The aggregation or accumulation of ABri peptide is considered to cause diseases such as familial British dementia and the like, and the accumulation site thereof is cerebrovascular amyloid.

In the present invention, being in an “equilibrium state” means that the rate of dissolution of soluble peptide, soluble oligopeptide, soluble polypeptide or soluble protein from insoluble fibril or aggregate of peptide, oligopeptide, polypeptide or protein into a solvent is the same as the rate of aggregation of soluble peptide, oligopeptide, polypeptide or protein in the solvent to form insoluble fibril or aggregate, and they are balanced.

The step of the identification method of the present invention may include simply measuring, in the presence of a test compound, the concentration of soluble peptide, soluble oligopeptide, soluble polypeptide or soluble protein dissolved from fibril or aggregate in a solvent.

In the present invention, by the fibril is meant a structure of about 1 μm in diameter where peptide and the like are linearly linked, and the aggregate means an assemblage of plural fibrils. The fibril and aggregate may be formed in vitro or in vivo. In consideration of the efficiency, operability, reproducibility of the test and the like of identification of a drug candidate, however, the fibril and aggregate formed in vitro is preferable.

In the present invention, by “being removed” means removal of soluble peptide, oligopeptide, polypeptide or protein from fibril or aggregate of aggregated peptide, oligopeptide, polypeptide or protein by dissolution.

For preparation of a sample in an equilibrium state, for example, soluble peptide, oligopeptide, polypeptide or protein, or fibril or aggregate thereof is added to a solvent and stood at an appropriate temperature (generally 0-40° C.) until the equilibrium state is achieved. Arrival at the equilibrium state can be confirmed by measuring the concentration of soluble peptide, oligopeptide, polypeptide or protein in a solvent chronologically. When the equilibrium state is achieved, a test compound is added to the reaction system and the mixture is incubated at an appropriate temperature (generally about 37° C.) (preferably for 1 day). After centrifugation at an appropriate temperature (generally 0-40° C.) for 1-60 min, the obtained supernatant is subjected to an analysis.

For preparation of a sample in a non-equilibrium state, for example, peptide, oligopeptide, polypeptide or protein is added to a solvent, and the mixture is incubated at a suitable temperature (generally 0-40° C.) for 1-6 hr to allow fibrillization or aggregation. When the fibrillization or aggregation is completed, the supernatant is separated by centrifugation at an appropriate temperature (generally 0-40° C.) for 1-60 min and the like. Then, a test compound is added to the obtained supernatant, and the mixture is incubated at a suitable temperature (generally about 37° C.) preferably for one day. After centrifugation at an appropriate temperature (generally 0-40° C.) for 1-60 min, the supernatant is collected and subjected to an analysis.

The solvent to be used here for the method of the present invention is not particularly limited as long as it does not prevent the object of the present invention and, for example, buffers such as PBS, acetate buffer, citrate buffer, Tris-HCl buffer and the like are used. The solvent is preferably adjusted to about pH3-8, more preferably about pH6-8 from the aspect of physiological conditions.

The concentration of soluble peptide, oligopeptide, polypeptide or protein in a solvent can be measured by a method known per se. For example, it can be measured by methods such as ELISA, Western blot, mass analysis, size-exclusion chromatography (SEC) and the like.

When, as a result of the above-mentioned measurement, an increase in the concentration of soluble peptide, oligopeptide, polypeptide or protein in a solvent can be confirmed by the addition of the test compound, as compared to the absence of the test compound, the test compound can be determined to be a drug candidate capable of removing peptide, oligopeptide, polypeptide or protein from fibril or aggregate. The thus-obtained compound can be used for various purposes and useful for the development of, for example, a drug for the prophylaxis or treatment of a disease caused by the aggregation of peptide, oligopeptide, polypeptide or protein or a reagent for studying the disease. As such disease, for example, Alzheimer's disease (AD), Parkinson's syndrome (PD), Huntington chorea, prion disease, Down's syndrome, Lewy body dementia, multiple system atrophy, Creutzfeldt-Jakob disease, Gerstmann-Sträussler syndrome, mad cow disease, spinobulbar muscular atrophy, spinocerebellar ataxia (SCA), dentatorubral-pallidoluysian atrophy (DRPLA) familial amyotropic lateral sclerosis retinitis, FTDP-17, progressive supranuclear palsy, corticobasal degeneration, Pick disease, familial British dementia or familial dementia accompanying neuroserpin inclusion bodies and the like can be mentioned. According to the present invention, a compound obtained by the identification method of the present invention is also provided.

According to the identification method of the present invention, a drug candidate capable of specifically removing peptide, oligopeptide, polypeptide or protein from fibril or aggregate of particular peptide, oligopeptide, polypeptide or protein can be obtained by appropriately selecting the kind of peptide, oligopeptide, polypeptide or protein. Such drug candidate can be useful for the prophylaxis or treatment of a disease caused by the aggregation of the particular peptide, oligopeptide, polypeptide or protein.

### <Ultrasonication>

The above-mentioned equilibrium state may be achieved under ultrasonication. Preferably, the ultrasonication does not substantially accompany heat generation. While the ultrasonication is not particularly limited, generally, 15 repeats of a 10 second ultrasonication under the conditions of 1-20 MHz, 0.1-10 W/cm², duty ratio 10-90% with a 10 second pause are performed, more preferably, 5 repeats of a 30 second ultrasonication under the conditions of 2.5-7.5 MHz, 0.5-5 W/cm², duty ratio 20-70% with a 10 second pause are performed, wherein the duty ratio means transmission time/(transmission time+transmission interval).

The above-mentioned ultrasonication promotes removal of peptide, oligopeptide, polypeptide or protein from fibril or aggregate. Therefore, ultrasonication accelerates arrival at the equilibrium state, and enables more rapid identification of a drug candidate.

Moreover, a method of removing peptide, oligopeptide, polypeptide or protein from fibril or aggregate by ultrasonication is also encompassed in the present invention.

Furthermore, the present invention encompasses a method for the treatment of a disease caused by aggregation of peptide, oligopeptide, polypeptide or protein, which comprises application of ultrasonication to a patient.

The present invention moreover provides an apparatus for treating a disease caused by the aggregation of peptide, oligopeptide, polypeptide or protein, which comprises a means for ultrasonicating an affected part to remove peptide, oligopeptide, polypeptide or protein from fibril or aggregate.

For ultrasonication here, generally, 15 repeats of a 10 second ultrasonication under the conditions of 1-20 MHz, 0.1-10 W/cm², duty ratio 10-90% with a 10 second pause are performed, more preferably, 5 repeats of a 30 second ultrasonication under the conditions of 2.5-7.5 MHz, 0.5-5 W/cm², duty ratio 20-70% with a 10 second pause are performed.

### <Dissolution Promoter>

The present invention provides a dissolution promoter for removing peptide, oligopeptide, polypeptide or protein from fibril or aggregate, which contains, as an active ingredient, a compound obtained by the above-mentioned identification method.

As is clear from the following Examples, as a compound obtained by the above-mentioned identification method, ferric dehydroporphyrin IX, amphotericin B, myricetin, tannic acid, curcumin, azure B and basic blue 41 are used.

Accordingly, the present invention provides a dissolution promoter for removing peptide, oligopeptide, polypeptide or protein from fibril or aggregate, which contains, as an active ingredient, at least one selected from ferric dehydroporphyrin IX, amphotericin B, myricetin, tannic acid, curcumin, azure B and basic blue 41.

Ferric dehydroporphyrin IX is a compound represented by the following chemical formula 1:

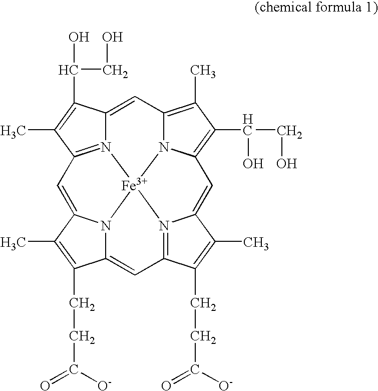

which is reported to inhibit in vitro the formation of a protease-resistant protein (PrP-res) and to be possibly useful for the treatment of transmissible spongiform encephalopathy (S. A. Priola et al., Science, 287, 1503 (Feb. 25, 2000)). There is also a report that the compound inhibits tau filament formation (S. Taniguchi et al., J Biol Chem, 280(9), 7614-7623, 2005).

Amphotericin B is a compound represented by the following chemical formula 2:

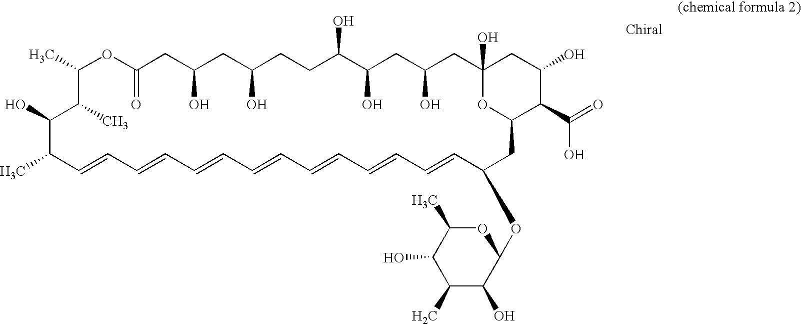

which is known to delay Aβ fiber formation (S. C. Hartsel et al., Biochemistry 42, 6228 (May 27, 2003)).

Myricetin and tannic acid are each a compound represented by the following chemical formulas 3 and 4, respectively:

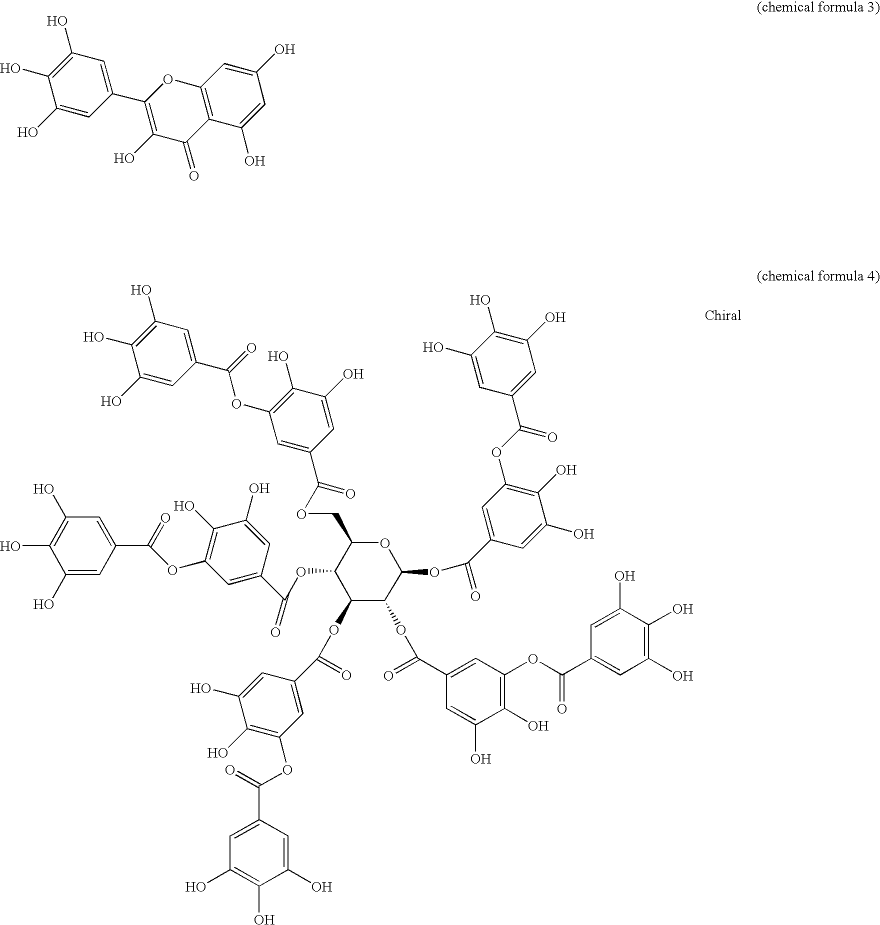

, which have been reported to show in vitro inhibitory action on the formation or elongation of Aβ (K. Ono, Biochim Biophys Acta 1690, 193 (Nov. 5, 2004).

Curcumin is a compound represented by the following chemical formula 5:

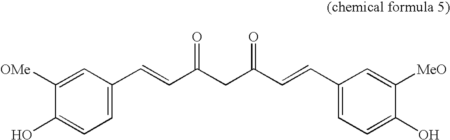

which has been reported to inhibit the formation of an Aβ (1-40) fiber (F. Yang et al., J Biol Chem, 280(7), 5892-5901, 2005), and is known to be effective as a therapeutic drug for Alzheimer (G. P. Lim et al., J Neurosci, 21(21):8370-7 (Nov. 1, 2001) and WO03/103583).

Azure B is a compound represented by the following chemical formula 6:

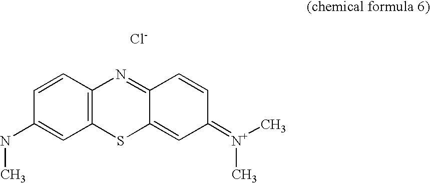

, which has been reported to inhibit tau filament formation (S. Taniguchi et al., J Biol Chem 280(9), 7614-7623, 2005).

Basic blue 41 is a compound represented by the following chemical formula 7:

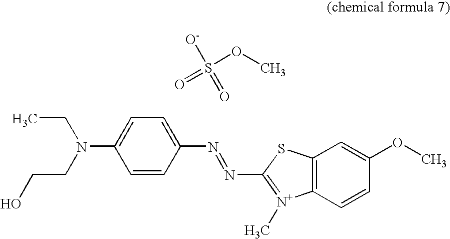

, which is one kind of thioflavine T, and thioflavine T is known to be a fluorescence indicator of amyloid fibers (H. Nakai et al, Lab Invest 62, 768 (June 1990)).

Since the dissolution promoter of the present invention can enhance the action to remove peptide, oligopeptide, polypeptide or protein from fibril or aggregate, it is useful for the prophylaxis or treatment of a disease caused by the aggregation of peptide, oligopeptide, polypeptide or protein.

As described in the aforementioned publications, the above-mentioned compounds are all known to be usable for various diseases. Since the compounds were newly found to be able to dissolve peptide, oligopeptide, polypeptide or protein from fibril or aggregate in the present invention, it is suggested that dissolution promoters containing these compounds can be used for the prophylaxis or treatment of diseases caused by the aggregation of peptide, oligopeptide, polypeptide or protein.

The dissolution promoter of the present invention can contain, in addition to the above-mentioned compound, optional carrier and the like. As the optional carrier, pharmaceutically acceptable carrier (mentioned later) can be mentioned.

The dissolution promoter of the present invention can be used as a pharmaceutical agent or a test reagent and the like, or used in vivo or in vitro. When the dissolution promoter of the present invention is used as a pharmaceutical agent, it can be orally or parenterally administered to animals (e.g., mammals such as human, simian, bovine, horse, dog, cat, rabbit, rat, mouse and the like), and can be used in the dosage form of oral administration agents such as tablet, capsule, troche, granule, powder and the like or in the dosage form of external preparations (liquid, lotion, suspension, emulsion, cream, ointment, gel etc.), injection, suppository and the like.

As a pharmaceutically acceptable carrier, for example, excipients such as sucrose, starch, mannit, sorbit, lactose, glucose, cellulose, talc, calcium phosphate, calcium carbonate and the like, binders such as cellulose, methylcellulose, hydroxypropylcellulose, polypropylpyrrolidone, gelatin, gum arabic, polyethylene glycol, sucrose, starch and the like, disintegrants such as starch, carboxymethylcellulose, hydroxypropyl starch, sodium glycol starch, sodium hydrogencarbonate, calcium phosphate, calcium citrate and the like, lubricants such as magnesium stearate, aerosil, talc, sodium lauryl sulfate and the like, aromatic such as citric acid, menthol, ammonium glycyrrhizinate, glycine, orange powder and the like, preservatives such as sodium benzoate, sodium bisulfite, methylparaben, propylparaben and the like, stabilizers such as citric acid, sodium citrate, acetic acid and the like, suspending agents such as methylcellulose, polyvinylpyrrolidone, aluminum stearate and the like, diluents such as surfactant and the like, dispersing agent, water, saline, orange juice and the like, base wax such as cacao butter, polyethylene glycol, white kerosene and the like, and the like can be mentioned, though not limited to these.

The preparation preferable for oral administration includes liquids obtained by dissolving an effective amount of a substance in a diluting solution such as water, saline and orange juice, capsule containing an effective amount of a substance as solid or granule, sachet or tablet, suspension wherein an effective amount of a substance is suspended in a suitable dispersion, an emulsion wherein a solution containing an effective amount of a substance is dispersed and emulsified in a suitable dispersion medium and the like.

As a preparation preferable for parenteral administration (e.g., subcutaneous injection, muscular injection, topical injection, intraperitoneal administration and the like), aqueous and non-aqueous isotonic sterile injections can be mentioned, which may contain antioxidant, buffer, antibacterial agent, isotonicity agent and the like. In addition, aqueous and non-aqueous sterile suspensions can be mentioned, which may include suspension, solubilizer, thickener, stabilizer, preservative and the like. The preparation can be sealed in a container by a unit dose or plural doses like ampoules and vials. In addition, an active ingredient and a pharmaceutically acceptable carrier may be lyophilized and preserved such that they only need to be dissolved or dispersed in a suitable sterile vehicle immediately before use.

While the dose of the dissolution promoter of the present invention cannot be said in general, since it varies depending on the activity and kind of the active ingredient, severity of the disease, the animal species to be the subject of administration, and drug acceptability, body weight, age and the like of the subject of administration, it is generally about 0.001-about 5 mg/kg/day for an adult based on the amount of the active ingredient.

### EXAMPLES

The present invention is explained in detail in the following by referring to Examples, which are not to be construed as limitative.

### Example 1

### (I) Method

### (1) Reagent and Solvent

Unless otherwise specified, the drug was obtained from Sigma Ltd. or Wako.

Water was distilled twice, and deionized using a Milli-Q system (Millipore Corporation, Bedford, Mass.).

### (2) Peptide

Aβ (1-40) and Aβ (1-42) were obtained from PEPTIDE INSTITUTE, INC. (Osaka, Japan). Aβ solution was prepared anew as described in Hartley, Walsh et al. 1999, for every experiment. Aβ (1-40) peptide (0.55 mg) was dissolved in 1 mM NaOH (130 μl) containing 0.01% phenol red.

10 mM NaOH was added to adjust the solution to pH 7.5. To give a 500 μM Aβ solution, PBS/ddH20 was added. Undissolved Aβ was removed by centrifugation before separating the supernatant (15,000×g, 3 min).

### (3) Sample Preparation

The 500 μM Aβ solution (20 μl) was taken in a 1.5 ml Eppendorf tube. The sample was incubated at 37° C. for several time periods until completion of fibrillization. After centrifugation at 22° C. (15,000×g, 10 min), the supernatant was removed.

The obtained pellets (i.e., Aβ fibril) were washed 3 times with phosphate buffer (PBS). Fresh PBS (20 μl) was carefully and gradually added to the pellets. The sample was incubated at 37° C. for one day. After centrifugation at 22° C. (15,000×g, 10 min), the supernatant (16 μl) was taken and subjected to the analysis. The remaining supernatant was removed. Fresh PBS (20 μl) was carefully and gradually added to the pellets.

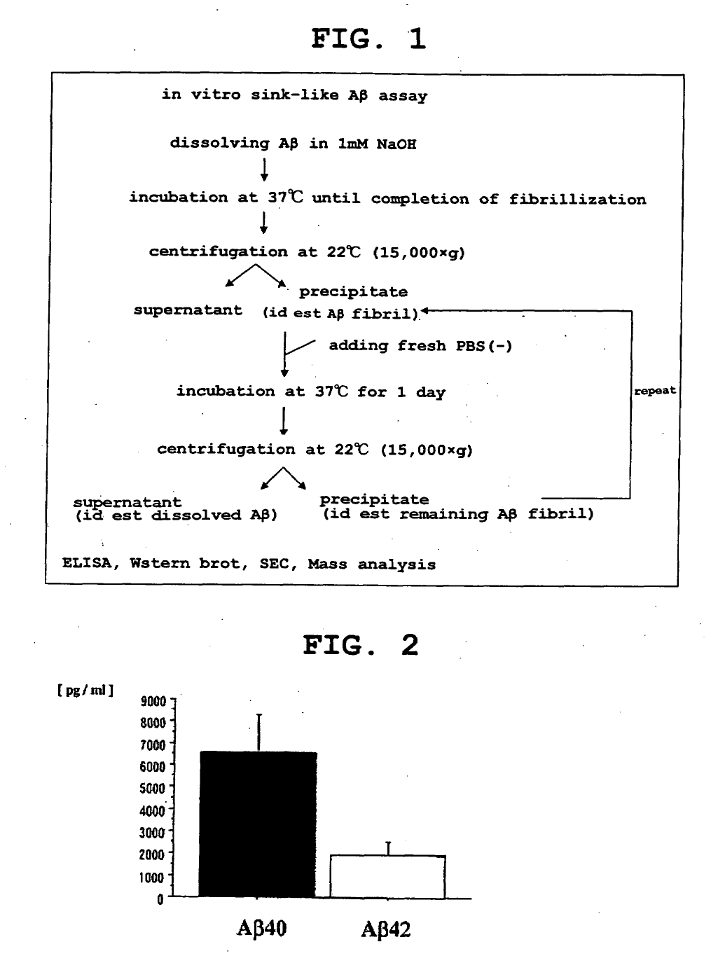

The above operation was repeated (see FIG. 1).

The sample was analyzed as in the following by ELISA, Western blot, mass analysis and size-exclusion chromatography (SEC).

### (4) Sandwich Method Aβ ELISA

Sandwich ELISA for Aβ (1-40) and Aβ (1-42) was performed using an Aβ (1-40) ELISA kit and Aβ (1-42) ELISA kit (Biosoure, IBL, Signet Laboratories and the like).

### (5) SEC

Superose 6 column (Amersham) was set to the HPLC system consisting of an injector and UV detector. After elution from the column at 0.04 ml/min, peptide was detected by UV absorption at 218 nm.

Each experiment was performed at least twice. Prepacked column was washed with 2N NaOH between experiments.

For respective studies, an appropriate column was equilibrated with an elution buffer in an amount corresponding to at least 3-fold column volume, and then calibrated with 5 kinds of molecular weight standard product: chicken ovalbumin (44,000); horse myoglobulin (17,000); human γ-globulin (158,000); bovine siloglobulin (670,000); and vitamin B₁₂ (1,350).

As the elution buffer, 75 mM NaCl, 5 mM Tris-HCl (pH 7.4) were used.

### (6) Gel Electrophoresis and Western Blot

Using Tris/Tricine gel (Invitrogen), SDS polyacrylamide gel electrophoresis (SDS-PAGE) was performed. A sample was mixed with a 2×SDS sample buffer (Invitrogen), and rapidly boiled for 5 min prior to electrophoresis. The sample was transferred onto a polyvinylidene difluoride (PVDF) membrane (Millipore Corporation, Bedford, Mass., US).

Western blot was performed using 6E10 antibody.

ECL was used as the detection system.

### (7) Mass Analysis

Analysis of Aβ species by combined immunoprecipitation/matrix associated laser desorption ionization-time of flight (MALDI-TOF) mass spectrometry (IP/MS) was performed as described (Okochi, Steiner et al. 2002).

### (8) Ultrasonication

A sample tube was exposed to ultrasonication (2.5 W/cm²) at room temperature for 30 seconds (5 repeats of 30 sec ultrasonication).

### (II) Results

Soluble Aβ dissolved from aggregated Aβ.

### (1) Sandwich ELISA for Dissolved Aβ (1-40) and Aβ (1-42)

Sandwich ELISA for dissolved Aβ (1-40) and Aβ (1-42) was performed according to the described experiment method. The amount of the dissolved Aβ (1-40) was 6562.5±1750.9 (S.E.) pg/ml (n=6). The amount of the dissolved Aβ (1-42) was 1939.7±607.6 (S.E.) pg/ml (n=6). The respective results are shown in FIG. 2.

### (2) Mass Analysis of Dissolved Aβ (1-40) and Aβ (1-42)

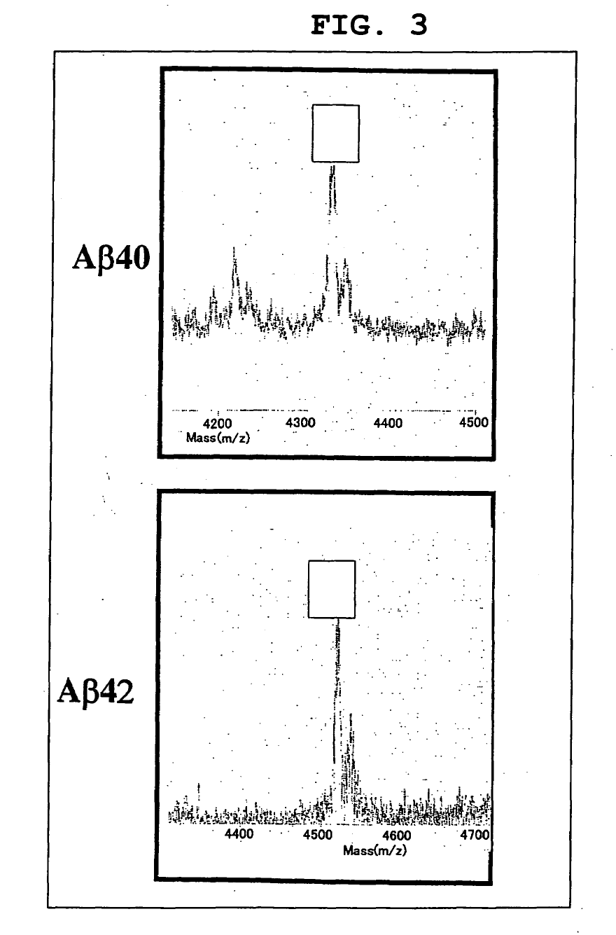

The molecular weight of dissolved Aβ (1-40) and Aβ (1-40) was confirmed to be 4329.8 and 4514.0, respectively, by mass analysis (FIG. 3).

### (3) Western Blot of Dissolved Aβ (1-40)

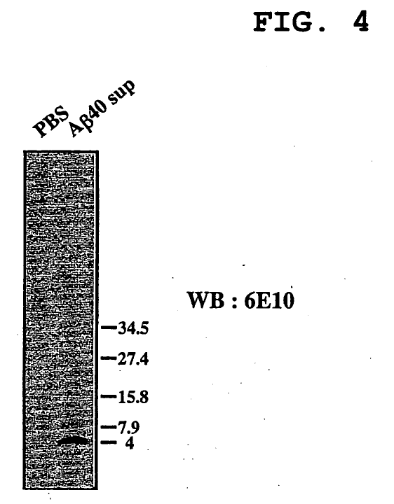

Detection of a nearly single band of low molecular weight is shown (FIG. 4).

### (4) SEC Analysis of Dissolved Aβ (1-40) and Aβ (1-42)

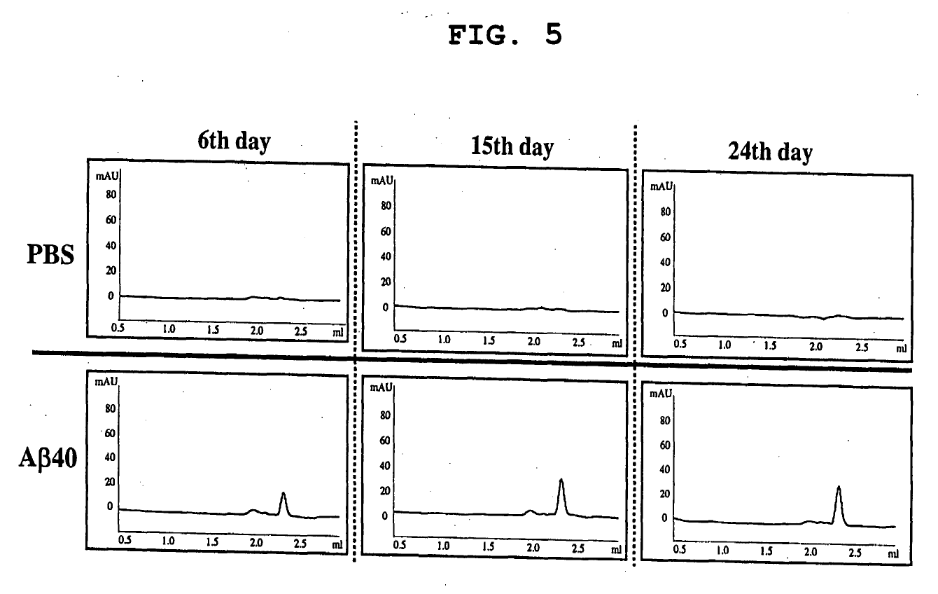

SEC analysis showed a sufficient single 2.4 ml peak (FIG. 5).

### (5) Effect of Ultrasonication

Ultrasonication promoted Aβ dissolution.

A sample tube was exposed to ultrasonication (2.5 W/cm²) at room temperature, duty ratio 20% for 30 seconds (5 repeats of 30 sec ultrasonication).

Sandwich ELISA for dissolved Aβ (1-40) and Aβ (1-42) was performed.

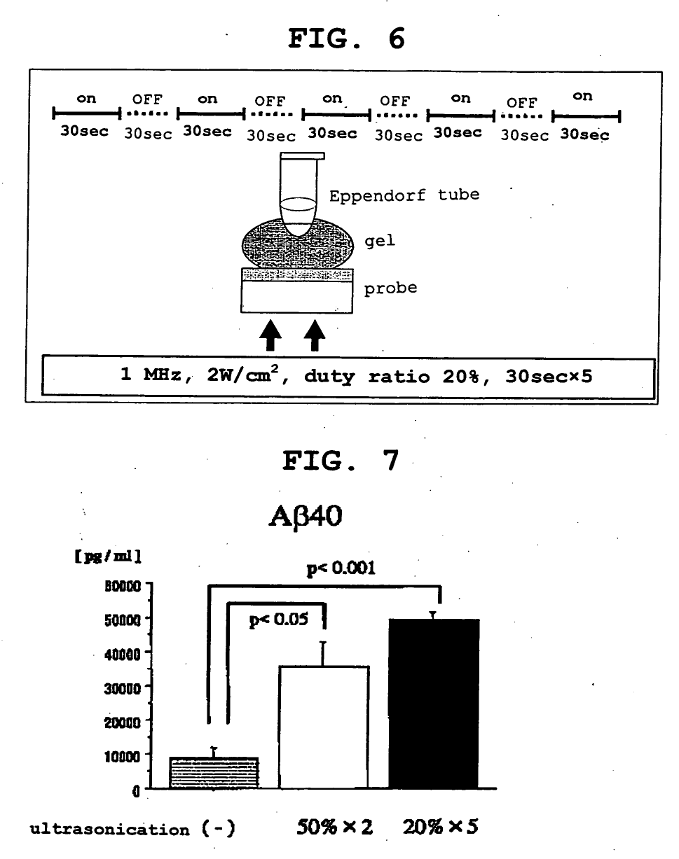

Ultrasonication for 30 seconds (5 repeats of 30 second ultrasonication at 2.5 W/cm², duty ratio 20%, room temperature) promoted dissolution of Aβ (1-40) (FIG. 6).

(control group: 8791.7±3121.1 (S.E.) pg/ml (n=3);

ultrasonication group: 49479.3±2167.6 (S.E.) pg/ml (n=3); less than p 0.001)

In another experiment method at duty ratio 50% (30 seconds, 2.5 W/cm², room temperature, 2 repeats of 30 second ultrasonication), dissolution of Aβ (1-40) was promoted.

(control group: 8791.7±3121.1 (S.E.) pg/ml (n=3); ultrasonication group 50%×twice: 35520±7140 (S.E.) pg/ml

(n=3); p less than 0.05)

The effect of ultrasonication is shown in FIG. 7.

### (6) SEC Analysis of Aβ (1-42) Fibrillization and Oligomerization

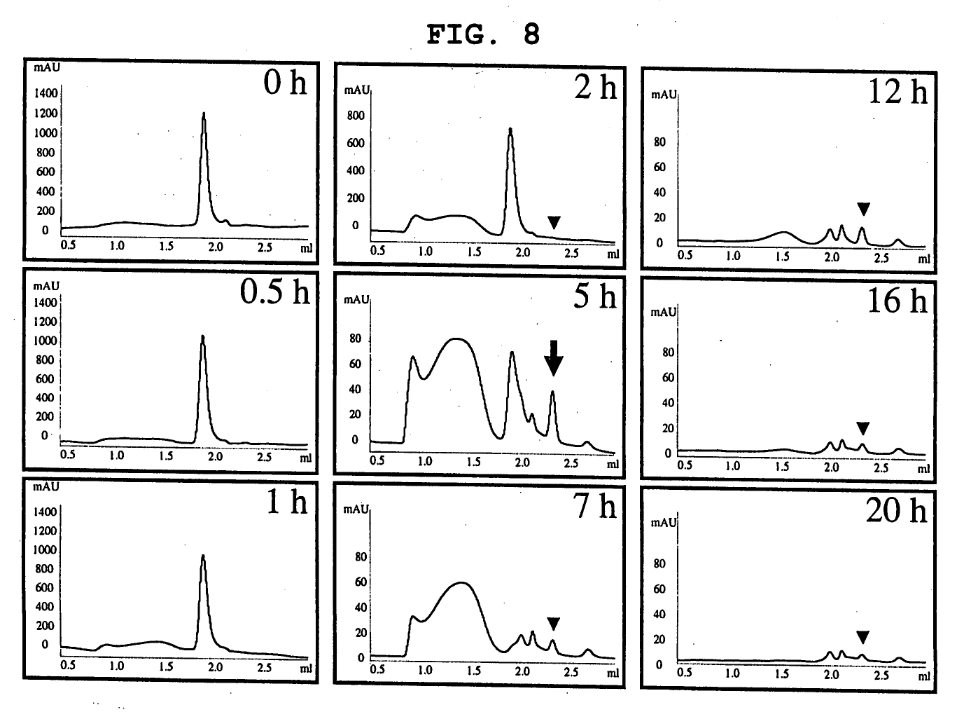

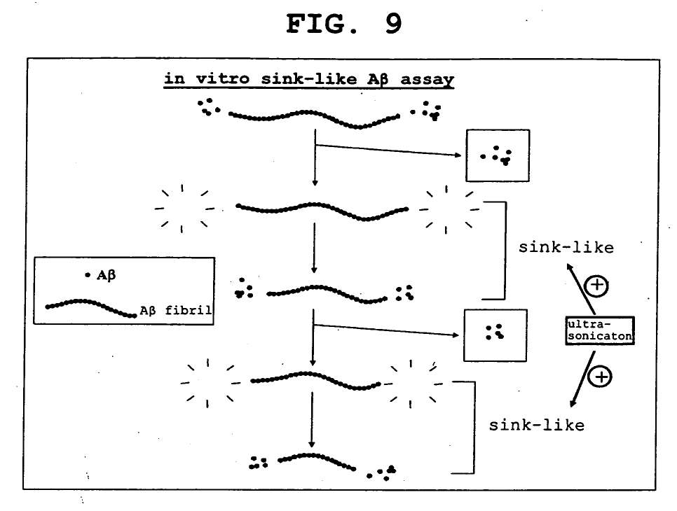

After dissolution, soluble Aβ (1-42) was immediately subjected to Superose 6 chromatography analysis by the experiment method as described. The results are shown in FIG. 8.

A peak derived from the gel (gel-included peak) was eluted at 1.9 ml.

The Aβ oligomer size in the peak could not be determined.

A large oligomer and a fibril precursor were observed during the time between 1 h and 12 h (arrow or triangle in the Figure).

The peak at 2.4 m increased at 5 h (arrow in the Figure).

Since this peak was observed when the dissolution of Aβ (1-40) was analyzed by SEC, it was eluted in the same fraction.

### Example 2

### (I) Method

### (1) Reagent and Solvent

Ferric dehydroporphyrin IX, amphotericin B, myricetin, tannic acid, curcumin, azure B and basic blue 41 were each obtained from Sigma-Aldrich.

Except those mentioned above, the preparation followed Example 1.

### (2) Peptide

Peptide was prepared in the same manner as in Example 1, (I)-(2)

### (3) Sample Preparation

A 500 μM Aβ (1-42) solution (20 μl) was separated in a 1.5 ml tube. The peptide was incubated for several days at 37° C. until fibrillization was completed. After centrifugation at 22° C. (15,000×g, 10 min), the supernatant was removed. The obtained pellets were gradually and carefully washed 3 times with PBS. Fresh PBS (20 μl) was carefully and gradually added to the pellets. The sample was incubated at 37° C. for one day. After centrifugation (15,000×g, 10 min) at 22° C., the supernatant was removed. Then, fresh PBS (20 μl) containing a compound: ferric dehydroporphyrin IX, amphotericin B, myricetin, tannic acid, curcumin, azure B or basic blue 41, or fresh PBS (20 μl) without any compounds as a control were carefully and gradually added to the pellets. The sample was incubated at 37° C. for one day. After centrifugation (15,000×g, 10 min) at 22° C., the supernatant (16 μl) (Aβ dissolved from fibril) was taken and subjected to Aβ ELISA analysis.

### (II) Results

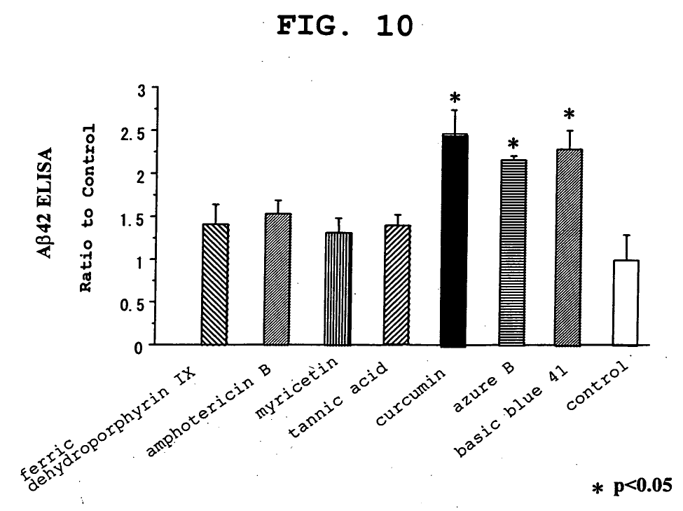

The results are shown in Table 1 and FIG. 10.

TABLE 1

| | average | SE |
|---|---|---|
| ferric dehydroporphyrin IX | 1.4124 | 0.224152 |
| amphotericin B | 1.5309 | 0.161446 |
| myricetin | 1.3196 | 0.16005 |
| tannic acid | 1.3969 | 0.123811 |
| curcumin | 2.4691 | 0.282381 |
| azure B | 2.1701 | 0.044954 |
| basic blue 41 | 2.299 | 0.220559 |
| control | 1 | 0.303872 |

The average value shows the ratio when the control is 1.

As shown in Table 1 and FIG. 10, when the above-mentioned compound was added, significant dissolution of Aβ was observed.

### REFERENCES

1. DeMattos, R. B., K. R. Bales, et al. (2001). “Peripheral anti-A beta antibody alters CNS and plasma A beta clearance and decreases brain A beta burden in a mouse model of Alzheimer's disease.” Proc Natl Acad Sci USA 98(15): 8850-5.
2. Hartley, D. M., D. M. Walsh, et al. (1999). “Protofibrillar intermediates of amyloid beta-protein induce acute electrophysiological changes and progressive neurotoxicity in cortical neurons.” J Neurosci 19(20): 8876-84.
3. Lombardo, J. A., E. A. Stern, et al. (2003). “Amyloid-beta antibody treatment leads to rapid normalization of plaque-induced neuritic alterations.” J Neurosci 23(34): 10879-83.
4. Matsuoka, Y., M. Saito, et al. (2003). “Novel therapeutic approach for the treatment of Alzheimer's disease by peripheral administration of agents with an affinity to beta-amyloid.” J Neurosci 23(1): 29-33.
5. Okochi, M., H. Steiner, et al. (2002). “Presenilins mediate a dual intramembranous gamma-secretase cleavage of Notch-1.” Embo J 21(20): 5408-16.
6. Schenk, D., R. Barbour, et al. (1999). “Immunization with amyloid-beta attenuates Alzheimer-disease-like pathology in the PDAPP mouse.” Nature 400(6740): 173-7.

### INDUSTRIAL APPLICABILITY

As is clear from the foregoing explanation, the present invention enables identification of a drug candidate capable of removing peptide, oligopeptide, polypeptide or protein from fibril or aggregate, by which the present invention is expected to be highly useful for the treatment, prophylaxis or study of the diseases caused by the aggregation of peptide, oligopeptide, polypeptide or protein, such as Alzheimer's disease and the like, which have various repercussions in the modern society.

This application is based on a patent application No. 2004-107746 filed in Japan, the contents of which are incorporated in full herein by this reference.

### SEQUENCE LISTING FREE TEXT

### SEQ ID NO: 1: Aβ (1-40)

SEQ ID NO: 2: Aβ (1-42)

1. A method of identifying a drug candidate capable of removing peptide, oligopeptide, polypeptide or protein from fibril or aggregate, which comprises measuring, in the presence of a test compound, the concentration of a soluble peptide, a soluble oligopeptide, a soluble polypeptide or a soluble protein in an equilibrium state in a solvent.

2. The method of claim 1, wherein the drug candidate is used for the treatment of a disease caused by the aggregation of peptide, oligopeptide, polypeptide or protein.

3. The method of claim 2, wherein the disease is selected from the group consisting of Alzheimer's disease (AD), Parkinson's syndrome (PD), Huntington chorea, prion disease, Down's syndrome, Lewy body dementia, multiple system atrophy, Creutzfeldt-Jakob disease, Gerstmann-Sträussler syndrome, mad cow disease, spinobulbar muscular atrophy, spinocerebellar ataxia (SCA), dentatorubral-pallidoluysian atrophy (DRPLA), familial amyotropic lateral sclerosis retinitis, FTDP-17, progressive supranuclear palsy, corticobasal degeneration, Pick disease, familial British dementia and familial dementia accompanying neuroserpin inclusion bodies.

4. The method of claim 1, wherein the fibril or aggregate was formed in vitro.

5. The method of claim 1, wherein the equilibrium state is achieved under ultrasonication.

6. The method of claim 5, wherein the ultrasonication is substantially unaccompanied by heat generation.

7. The method of claim 5, wherein the ultrasonication conditions include 5 repeats of a 30 second ultrasonication at 1 MHz, 2 W/cm2, duty ratio 20% with a 10 second pause.

8. A method of identifying a drug candidate capable of removing β-amyloid (Aβ) from fibril or aggregate formed in vitro, which comprises measuring, in the presence of a test compound, the concentration of soluble β-amyloid (Aβ) in an equilibrium state in a solvent.

9. The method of claim 8, wherein the fibril or aggregate consists of Aβ (1-40).

10. The method of claim 8, wherein the fibril or aggregate consists of Aβ (1-42).

11. The method of claim 8, wherein the equilibrium state is achieved under ultrasonication.

12. The method of claim 11, wherein the ultrasonication is substantially unaccompanied by heat generation.

13. The method of claim 12, wherein the ultrasonication conditions include 5 repeats of a 30 second ultrasonication at 1 MHz, 2 W/cm2, duty ratio 20% with a 10 second pause.

14. A treatment method of a disease caused by aggregation of peptide, oligopeptide, polypeptide or protein, which comprises application of ultrasonication to a patient.

15. The method of claim 14, wherein the disease is selected from the group consisting of Alzheimer's disease, Parkinson's syndrome, Huntington chorea, prion disease, Down's syndrome, Lewy body dementia, multiple system atrophy, Creutzfeldt-Jakob disease, Gerstmann-Sträussler syndrome, mad cow disease, spinobulbar muscular atrophy, spinocerebellar ataxia (SCA), dentatorubral-pallidoluysian atrophy (DRPLA), familial amyotropic lateral sclerosis retinitis, FTDP-17, progressive supranuclear palsy, corticobasal degeneration, Pick disease, familial British dementia and familial dementia accompanying neuroserpin inclusion bodies.

16. A dissolution promoter for removing peptide, oligopeptide, polypeptide or protein from fibril or aggregate, which comprises, as an active ingredient, the compound obtained by the method described in claim 1.

17. A dissolution promoter for removing peptide, oligopeptide, polypeptide or protein from fibril or aggregate, which comprises, as an active ingredient, at least one selected from the group consisting of ferric dehydroporphyrin IX, amphotericin B, myricetin, tannic acid, curcumin, azure B and basic blue 41.

18. A dissolution method comprising dissolving peptide, oligopeptide, polypeptide or protein from fibril or aggregate, by the use of the compound obtained by the method described in claim 1.

19. (canceled)

20. An apparatus for treating a disease caused by the aggregation of peptide, oligopeptide, polypeptide or protein, which comprises a means for ultrasonicating an affected part to remove peptide, oligopeptide, polypeptide or protein from fibril or aggregate.

21. A method of identifying a drug candidate capable of removing peptide, oligopeptide, polypeptide or protein from fibril or aggregate, which comprises measuring, in the presence of a test compound, the concentration of the soluble peptide, soluble oligopeptide, soluble polypeptide or soluble protein dissolved from fibril or aggregate in a solvent.
